SEQUENCE LISTING (2 sequences)

SEQ ID NO: 1 - protein, 328 aa, Thermococcus kodakarensis KOD1
MKLLVLGTGGTIASAKTEMGYKAALSADDILQLAGIRREDGAKIETRDILNLDSTLIQPEDWVTIGRAVFEAFDEYDGIVITHGTDTLAYTSSALSFMIRNPPIPVVLTGSMLPITEPNSDAPRNLRTALTFARKGFPGIYVAFMDKIMLGTRVSKVHSLGLNAFQSINYPDIAYVKGDEVLVRHKPRIGNGEPLFDPELDPNVVHIRLTPGLSPEVLRAVARATDGIVLEGYGAGGIPYRGRNLLEVVSETAREKPVVMTTQALYGGVDLTRYEVGRRALEAGVIPAGDMTKEATLTKLMWALGHTRDLEEIRKIMERNIAGEITGS

SEQ ID NO: 2 - DNA, 987 bp, Thermococcus kodakarensis KOD1
atgaaacttctggttctcggcacggggggcaccatagcgagtgcaaagacggagatgggatacaaggcagcgctcagtgcagatgatatccttcagctggcgggcattaggcgagaagacggtgccaagatagaaacccgggacatcctgaaccttgacagcactctcatacagccagaggattgggtgactatcggacgggcggtttttgaggcttttgatgaatacgacggtatagtaataacacacggaacagacacactcgcctacacttcttccgcccttagcttcatgataaggaatccgccgatacccgttgttctgaccggttcaatgctcccgataaccgagccaaacagcgacgcacccagaaaccttagaacggcactcacctttgcaaggaaaggatttcccgggatatacgtggctttcatggacaagataatgctcgggacgcgcgtttctaaggttcactccctcggcctcaacgcctttcagagtatcaattacccagacatagcctacgtgaagggtgacgaagtcctcgttagacataagcccagaattgggaatggtgaaccattgtttgatccagagcttgaccctaacgttgtccatatcagacttactccagggctttcaccggaggttcttagggcagtcgccagggccacagatggaatcgtcctcgagggttacggagcaggtggtatcccatacaggggcagaaatctcctggaggtagtctctgagacagcgagagaaaaaccggttgtcatgacgacgcaggctctctacgggggagttgatctgacgcgctatgaagttgggagaagggcccttgaagccggcgtgatcccagcgggcgacatgacaaaggaagcgactttgacaaagctcatgtgggcgctgggacacaccagagatcttgaagaaatcagaaaaattatggaacgaaacatcgcgggcgaaatcactgggagttaa

(19) United States
(12) Patent Application Publication
(10) Pub. No.: US 2014/0256002 A1
(43) Pub. Date: Sep. 11, 2014

(54) COMPOSITION FOR BREAKING DOWN L-ASPARAGINE COMPRISING L-ASPARAGINASE, AND PRODUCTION METHOD FOR L-ASPARAGINASE

(71) Applicants: Jae-Ho Shin, Daegu (KR); Sung Jun Hong, Daegu (KR); Yun Ha Lee, Daegu (KR)

(72) Inventors: Jae-Ho Shin, Daegu (KR); Sung Jun Hong, Daegu (KR); Yun Ha Lee, Daegu (KR)

(73) Assignee: KYUNGPOOK NATIONAL UNIVERSITY INDUSTRY-ACADEMIC COOPERATION FOUNDATION, Daegu (KR)

(21) Appl. No.: 14/239,069

(22) PCT Filed: Aug. 17, 2012
(86) PCT No.: PCT/KR2012/006570

§ 371 Date: May 22, 2014

(30) Foreign Application Priority Data
Aug. 19, 2011 (KR) 10-2011-0082821

(51) Int. Cl. C12P 13/20

(52) U.S. Cl. CPC C12P 13/20
USPC 435/109; 536/23.2; 435/229; 435/320.1; 435/252.33

(57) ABSTRACT

The present invention relates to a composition for breaking down L-asparagine comprising L-asparaginase, and to a production method for L-asparaginase. The L-asparaginase of the present invention differs from existing L-asparaginase in that it has improved heat stability and exhibits high activity even at high temperatures, and thus it improves upon shortcomings of existing L-asparaginase and so can be used to advantage industrially.

### TECHNICAL FIELD

The present invention relates to a composition for breaking down L-asparagine, comprising L-asparaginase, a method for producing L-asparaginase, and a method for breaking down L-asparagine using L-asparaginase.

### BACKGROUND ART

L-asparaginase is a deaminase that produces NH₃ and L-aspartic acid by breaking down L-asparagine. This enzyme is produced by various microorganisms and widely used in the fields of foods and pharmaceuticals.

As an example of use in the food industry, L-asparaginase is used to prevent Maillard reaction in foods. Acrylamide is produced in starchy foods that are baked in the oven or fried in oil. These foods contain high amounts of L-asparagine and sugar, which react with each other at high temperatures, and this process is referred to as the Maillard reaction. When the Maillard reaction takes place, it causes the surface of the food to turn brown or black. Therefore, when the food is treated with L-asparaginase, the Maillard reaction does not take place, thus reducing the production of acrylamide.

For medicine and medical supplies, L-asparaginase is used for the treatment of malignant tumors of lymphocytes and, in particular, is widely used for acute leukemia. Moreover, in the treatment of cancer cells, high amounts of L-asparagine are required when the malignant tumors grow, and thus L-asparaginase is used to degrade L-asparagine so as to inhibit the growth of malignant tumors, thus reducing or eliminating tumor cells.

L-asparaginase is produced by various microorganisms. In particular, L-asparaginase derived from Erwinia chrysanthemi and Escherichia coli is used medically. However, the thus prepared enzymes are very sensitive to pH or temperature, and thus deterioration of medicines is concerned during long-term storage or transportation.

Therefore, there is a need for the development of L-asparaginase having solved the above problems and having improved stability.

### DISCLOSURE

### Technical Problem

The present inventors have studied on L-asparaginase with less risk of deterioration and found that L-asparaginase produced from hyperthermophilic Thermococcus kodakarensis KOD1 exhibits its activity at different pH levels and temperatures and has excellent breakdown of L-asparagine, thus completing the present invention.

An object of the present invention is to provide a composition for breaking down L-asparagine, comprising L-asparaginase with improved thermal stability and optimal activity at high temperatures and a method for producing L-asparaginase.

Moreover, another object of the present invention is to provide a method for breaking down L-asparagine using the L-asparaginase.

### Technical Solution

To accomplish the above objects, the present invention provides a composition for breaking down L-asparagine, comprising L-asparaginase with improved thermal stability and a method for producing L-asparaginase.

Moreover, the present invention provides a method for breaking down L-asparagine using the L-asparaginase.

### Advantageous Effects

Unlike the conventional L-asparaginases, the L-asparaginase of the present invention has improved thermal stability and exhibits high activity at high temperatures and thus can be effectively used for industrial purposes by overcoming the drawbacks of the conventional L-asparaginases.

### DESCRIPTION OF DRAWINGS

FIGS. 1A and 1B show the homology of L-asparaginases derived from other strains with recombinant L-asparaginase derived from Thermococcus kodakarensis KOD1 of the present invention.

FIG. 2 shows that the recombinant L-asparaginase of the present invention has been highly purified.

FIG. 3 shows the change in activity of the recombinant L-asparaginase of the present invention with temperature.

FIG. 4 shows the change in activity of L-asparaginase with a change in pH (◯: treated with citrate-NaOH (pH 3-6.5); ▪: treated with sodium phosphate (pH 6-7); Δ: treated with HEPES-NaOH (pH 7-8.5); and ♦: treated with glycine-NaOH (pH 8.5-10)).

FIG. 5 shows the change in activity of L-asparaginase upon being heated (heat-treatment temperatures—: 60° C.; □: 80° C.; and ▴: 100° C.).

FIG. 6 shows the change in stability of L-asparaginase with a change in pH (: KCl—HCl (pH 1.5 and 2); □: glycine-HCl (pH 2 and 3); ▴: citrate-phosphate (pH 3, 4, 5 and 6); ∇: sodium phosphate (pH 6 and 7); ♦: HEPES-NaOH (pH 7, 8 and 8.5); ∘: glycine-NaOH (pH 8.5, 9, 10 and 11); ▪: sodium phosphate-NaOH (pH 12); and Δ: KCl—NaOH (pH 13)).

FIG. 7 shows the substrate affinity of L-asparaginase.

FIG. 8 shows the enzyme kinetics of L-asparaginase.

### BEST MODE FOR CARRYING OUT THE INVENTION

The present invention provides a composition for breaking down L-asparagine, comprising L-asparaginase represented by an amino acid sequence of SEQ ID NO: 1.

The L-asparaginase according to the present invention comprises a protein derived from Thermococcus kodakarensis KOD1 and having an amino acid sequence represented by SEQ ID NO: 1 and functional equivalents of the protein. The term “functional equivalents” refers to those having a sequence homology of 70% or higher, preferably 80% or higher, more preferably 90% or higher, most preferably 95% or higher with the amino acid sequence represented by SEQ ID NO: 1, as a result of addition, substitution or deletion of amino acids, and refers to proteins that exhibit substantially the same biological activity as the protein represented by SEQ ID NO: 1. The term “substantially the same biological activity” refers to having the activity of L-asparaginase.

The L-asparaginase of the present invention may be produced by recombination of gene sequences derived from hyperthermophilic Thermococcus kodakarensis KOD1.

The L-asparaginase of the present invention exhibits thermal stability even at high temperatures and may exhibit high activity at a temperature 70° C. to 100° C., and more preferably exhibit an optimal activity at a temperature 80° C. to 100° C.

Moreover, the L-asparaginase of the present invention may exhibit enzymatic activity at different pH ranges, such as pH 3 to 6.5 and pH 8.5 to 10, and preferably exhibit optimal enzymatic activity at a pH of 7 to 9.

The composition for breaking down L-asparagine of the present invention may be used as an additive to foods and medicines. In particular, it is useful as an additive to foods cooked at high temperatures. Moreover, the composition for breaking down L-asparagine of the present invention is a natural food ingredient, and thus it has no side effects in the body and can be used as an additive to various foods. Here, the types of foods which the composition of the present invention could be added are not particularly limited, and the amount of the composition of the present invention added to foods is not particularly limited, but it is preferably that the amount of the composition of the present invention added to foods is 1 to 10% (w/w).

The composition for breaking down L-asparagine of the present invention may include a gene encoding the amino acid sequence of SEQ ID NO: 1, and the gene may be a nucleotide sequence represented by SEQ ID NO: 2.

A gene encoding the L-asparaginase according to the present invention may be inserted into a suitable expression plasmid to transform a host cell. The gene sequence of the present invention may be operably linked to an expression control sequence, and the operably linked-gene sequence and expression control sequence may be included in one expression vector together with a selection marker and a replication origin. The term “operably linked” may refer to a gene and an expression control sequence linked in a manner to allow the gene expression when a suitable molecule binds to the expression control sequence. The term “expression control sequence” refers to a DNA sequence which controls the expression of a nucleic acid sequence operably linked in a specific host cell. The control sequence may comprise a promoter for performing transcription, an arbitrary operator sequence for controlling transcription, a sequence encoding a suitable mRNA ribosome binding site, and a sequence for controlling the termination of transcription and translation. A vector suitable to introduce the nucleic acid sequence of the gene according to the present invention may be selected by those skilled in the art, and any vectors that can introduce the L-asparaginase gene sequence into a host cell may be used in the present invention. The term “vector” refers to a nucleic acid molecule capable of transporting another nucleic acid to which it has been linked. The term “expression vector” is intended to include a plasmid, cosmid or phage, which can be used to synthesize a protein encoded by a recombinant gene carried by the vector. A preferred vector is a vector that can self-replicate and express a nucleic acid bound thereto.

As a specific example, the present invention provides a recombinant plasmid as an expression vector comprising the gene of SEQ ID NO: 2. Examples of the expression vectors may include pET21a(+), pWHM3, pHZ1080, pBW160, pMT3226, pANT1200, pANT1201, pANT1202, pANT849, pIJ4090, pIJ4123, pMT3206, and [symbol] pCZA185 (Hopwood, D. A., et al., Practical Streptomyces Genetics, The John innes Foundation Norwich, England, 267-268, 2000), and pET21a(+) (Novagen Inc, Hessen, Germany) was used in the present invention.

Moreover, the present invention provides a host cell transformed with the recombinant vector. The recombinant vector can be used to produce transformed cells capable of producing L-asparaginase with high yield by transforming cells such as prokaryotes, fungi, plant and animal cells. The term “transformation” means that foreign DNA or RNA is absorbed into cells to change the genotype of the cells. Known transformation methods for each cell type may be used to prepare the host cell, and in the present invention, E. coli was used in a preferred embodiment, but not limited thereto.

Furthermore, the present invention provides a method for producing L-asparaginase, comprising the steps of: (a) culturing a transformant for producing L-asparaginase, transformed with a recombinant plasmid containing a gene having a nucleotide sequence of SEQ ID NO: 2; and (b) isolating and purifying L-asparaginase from the transformant in step (a).

The transformant may be E. coli transformed by electroporation and may be cultured by placing a transformant, into which the nucleotide sequence of SEQ ID NO: 2 is introduced, in 1 ml S.O.C medium, culturing the transformant at 37° C. for 1 hour, and then plating the transformant on LB medium containing 10 μg/ml ampicillin.

The isolation and purification of L-asparaginase from the transformant may be performed by centrifugation of the culture medium, and the transformant may be heat-treated at 65° C. for 10 minutes to remove E. coli proteins. By the removal of non-targeted proteins, it is possible to obtain highly purified L-asparaginase.

Moreover, the present invention provides a method for breaking down L-asparagine, comprising the step of treating L-asparagine with L-asparaginase represented by an amino acid sequence of SEQ ID NO: 1.

The L-asparaginase may exhibit an optimal activity of breaking down L-asparagine at a temperature of 80° C. to 100° C. and at a pH of 7 to 9.

Therefore, unlike the conventional L-asparaginases, the L-asparaginase of the present invention exhibits high enzymatic activity with stability at high temperatures and at different pH ranges and thus can used as an effective breakdown enzyme.

### MODE FOR THE INVENTION

Hereinafter, the present invention will be described in detail with reference to the following Examples. However, the following Examples are intended only to illustrate the present invention, and the present invention is not limited by the following Examples

### Example 1

### Materials and Methods

1.1 Reagents

Reagents of the present invention were all guaranteed reagents (GR) purchased for their intended use. Restriction enzymes and other modification enzymes used for isolation and manipulation of DNA were purchased from Takara (Honshu, Japan) and Fermentas (Ontario, Canada). Plasmid DNA extraction kits were purchased from Solgent (Daejeon, Korea), and DNA polymerases, dNTPs, PCR buffers, etc. used in the polymerase chain reaction (PCR) were those purchased from Takara (Honshu, Japan) and those isolated and purified in the laboratory. Primers used in the PCR were purchased from Genotech (Daejeon, Korea). Protein purification of L-asparaginase was performed using Ni-Sepharose FF resin. Culture media of strains were mainly purchased from Difco (Missouri, USA), and antibiotics such as ampicillin, chloramphenicol, tetracycline, etc. were purchased from Sigma (Missouri, USA). The activity of L-asparaginase was measured using Nessler's reagent.

1.2 Strains and Plasmids

Escherichia coli XLI-Blue MRF′ was used for manipulation, storage and extraction of plasmids. E. coli Rosetta (DE3) was used for over-expression of L-asparaginase using a T7 promoter. Plasmid, called pRARE, is contained in E. coli Rosetta (DE3) and replaced with an amino acid that E. coli prefers during expression of heterologous proteins, thus facilitating the expression. T-blunt vector kits (Solgent, Daejeon, Korea) were used for DNA cloning in E. coli and subcloning for sequencing, and plasmid pET21a(+) (Novagen, Hessen, Germany) was used for the expression of recombinant proteins in E. coli.

1.3 Media and Culture Conditions

Luria Bertani (LB) medium was used for the growth and maintenance of E. coli and, if necessary, ampicillin antibiotic in a final concentration of 100 μg/ml was added to the medium. S.O.C. medium was used to increase the efficiency of transformation. In the case of liquid culture medium for the growth of E. coli, the liquid medium was inoculated with seed cultures containing E. coli 1% (v/v) grown in LB medium and shaking-cultured at 37° C. and 200 rpm. In the case of solid culture, LB agar medium was prepared and cultured in an incubator at 37° C. overnight.

### Example 2

### DNA Isolation and Manipulation

2.1 Isolation and Purification of Plasmid DNA

[Plasmid DNA was isolated by alkali-lysis method (Bimboim and Doly, 1979). E. coli harboring plasmids was cultured overnight in LB medium containing 100 μg/ml ampicillin and then cells were harvested by centrifugation (5,000 g, 15 minutes).

The harvested cells were suspended in TEG buffer (25 mM Tris. Cl, 50 mM glucose, 10 mM EDTA, pH 8.0) and reacted at room temperature for 5 minutes. 2 volumes (v/v) of 1% sodium dodecyl sulfate (SDS)-0.2N NaOH solution was added to the TEG buffer and dissolved at room temperature for 10 minutes. 1.5 volumes (v/v) of 3 M potassium acetate (pH 5.2) was added to the dissolved solution, left in ice for 10 minutes, and then centrifuged (5,000 g, 20 minutes).

The supernatant obtained by the centrifugation was transferred to a new centrifugation tube. Then, 0.6 volumes (v/v) of isopropanol was added to the supernatant to precipitate DNA, left at room temperature for 10 minutes, and then centrifuged (5,000 g, 15 minutes). The supernatant was discarded, and the precipitate was washed with 70% (v/v) ethanol, and then centrifuged (5,000 g, 5 minutes). The precipitated plasmid DNA was dissolved in 3 ml TE buffer (10 mM Tris-Cl, 1 mM EDTA, pH 8.0).

High purity plasmid DNA was purified by polyethylene glycol (PEG) precipitation method (Sambrook and Russell, 1989). To remove RNA from the plasmid DNA solution, an equivalent amount (v/v) of 5 M LiCl was added to the plasmid DNA solution, left in ice for 20 minutes, and then centrifuged (12,000 g, 10 minutes, 4° C.). An equivalent amount of isopropanol was added to the supernatant, left at −20° C. for 20 minutes, and then centrifuged (12,000 g, 10 minutes). The supernatant was discarded, and the precipitate was dissolved in 500 μl TE buffer (pH 8.0). To remove RNA remaining in the solution, the solution was treated with 20 μg/ml RNase A and reacted at room temperature for 30 minutes. The solution was treated with an equivalent amount of 30% (w/v) PEG (Hw 6,000)/NaCl solution, left to stand at room temperature for 1 hour, and then centrifuged (12,000 g, 10 minutes, 4° C.). 400 μl TE buffer (pH 8.0) was added, an equivalent amount of PCI (phenol:chloroform:isoamylalcohol=25:24:1) was added, vigorously mixed, and then centrifuged (12,000 g, 10 minutes, 4° C.). The isolated supernatant was transferred to a new E-tube, 0.1 volumes (v/v) of 3 M sodium acetate (pH 5.2) and 2 volumes (v/v) of ethanol were added, DNA was precipitated at −20° C., and then centrifuged (12,000 g, 10 minutes). 50% (v/v) ethanol was added, centrifuged, and dissolved in 400 μl TE buffer (pH 8.0). The extracted plasmid DNA was analyzed by electrophoresis, and the purity of plasmid DNA was determined when the ratio of absorbances measured at A260 and A280 was 1.8. The purified plasmid DNA was used for genetic recombination. Plasmid DNA prepared for sequencing was extracted and purified using a plasmid mini-prep kit (Solgent, Korea).

2.2 Preparation of E. coli Competent Cells

E. coli competent cells for transformation were prepared by modifying the method of Hanahan (1983). A single colony of E. coli was placed in 5 ml LB medium, cultured at 37° C. and 200 rpm overnight, inoculated into new 500 ml of 1% (v/v) LB medium, and cultured at 37° C. and 200 rpm, and the culture medium was taken out when the OD₆₀₀ reached 0.5 to 0.6 and left in ice for 5 minutes. The culture medium was centrifuged (5,000 g, 20 minutes, 4° C.) and cells were harvested at the initial exponential phase. The harvested cells were washed two times with 10% (v/v) glycerol solution. The harvested cells were mixed with 10% (v/v) glycerol in the same amount as the harvested cells, each 100 μl of harvested cells was placed in an E-tube, kept at −70° C., and used for the transformation of plasmid DNA.

2.3 Transformation of Recombinant Plasmids

E. coli was transformed by modified electroporation (Dower et al., 1988). 0.2 cm cuvette manufactured by Bio-Rad (NY, USA) was used and left in ice for 5 minutes before electroporation. 100 μl competent cells and 2 to 4 μl plasmid DNA were mixed in the cuvette and subjected to electroporation at 2.5 kV and 200Ω. Then, 1 ml S.O.C medium was added, and the cells were incubated at 37° C. for 1 hour and plated on LB medium containing 100 μg/ml ampicillin. The medium was incubated at 37° C. overnight to identify transformed cells. The transformed cells were screened on LB medium containing 10 mg/ml X-gal (bromo-chloro-indolyl-galactopyranoside) and 40 mM IPTG (isopropyl β-D-1-thiogalactopyranoside) by Blue/White colony selection (Sambrook and Russell, 1989), if necessary.

2.4 DNA Electrophoresis and Quantification

DNA electrophoresis was performed by the Ausubel's method (1992). A 0.8% agarose gel was used in the DNA electrophoresis and the experiment was performed at different concentrations of agarose gel, if necessary. The agarose gel was electrophoresed at a voltage of 15 mV per cm using 0.5× TAE buffer (40 mM Tris-acetate, 1 mM EDTA) for 20 minutes, treated with 0.5 mg/ml ethidium bromide for 20 minutes to stain the DNA, and subjected to UV irradiation, thus observing the DNA. DNA was quantified by comparing the relative intensity using 0.5 μg/5 μl λ/Hind III DNA used as a marker or quantified using a Nanophotometer from Implen (California, USA) at 260 nm.

2.5 DNA Cleavage and Ligation

DNA cleavage was performed in reaction solutions and at reaction temperatures for restriction enzymes according to the manufacturer's instruction (Fermentas, Ontario, Canada) for each restriction enzyme. Cleaved plasmid DNAA fragments were collected using a gel extraction kit (Bioneer, Daejeon, Korea). For ligation of isolated DNA fragments into plasmid DNA, the cleaved plasmid DNA and the isolated DNA were reacted at a ratio of 1:3 or 1:4 at 25° C. for 1 hour using T4 DNA ligase (Fermentas, Ontario, Canada) to ligate the DNA fragments, and transformed into E. coli, obtaining a large amount of plasmid DNA.

### Example 3

### DNA Amplification and Sequencing

3.1 Synthesis of Primer

Primer design of L-asparaginase according to the present invention was performed by Genotech (Daejeon, Korea) based on the genomic sequence of Thermococcus kodakarensis KOD1 registered in National center for biotechnology information (NCBI). Moreover, the primer was designed such that a 6× his-tag was added to the C-terminus of a protein for protein purification using Ni-NTA affinity chromatography (TK1656 N-terminus primer: 5′-CGGGATCCCATATGAAACTTCTGGTTCTCG-3′; TK1656 C-terminus TEV primer 5′-CTGAAAGTACAGGTTCTCACTCCCAGTGATTTCGCC-3′).

3.2 PCR Conditions

PCR was performed using a PCR reaction mixture containing 10×Pfu DNA polymerase buffer (200 mM Tris-HCl (pH 8.8), 100 mM (NH₄)₂SO₄, 100 mM KCl, 1% (v/v) Triton X-100, 1 mg/ml BSA, 20 mM MgSO₄) 5 μl, 2.5 mM dNTP 2 μl, template DNA 1 μl (10 ng/μl), 10 pmol forward primer and 10 pmol reverse primer 1 μl, dimethyl sulfoxide (DMSO) 5 μl, dUTPase 1 μl, pfu DNA polymerase 1 μl, sterilized distilled water 37 μl. The reaction was carried out for 30 cycles with denaturation at 98° C. for 2 minutes and at 96° C. for 30 seconds, annealing at 54° C. for 30 seconds, and extension at 72° C. for 1 minute. The PCR products were analyzed on a 8% agarose gel (w/v). The PCR products were purified using a PCR purification kit (Solgent, Daejeon, Korea).

3.3 DNA Sequencing

The PCR products were cloned into the T-Blunt vector, and the DNA sequencing was performed by Solgent (Daejeon, Korea). Homology of the DNA sequences was analyzed using BLAST (http://www.ncbi.nlm.nih.gov/BLAST/blast.cgi) of the National center for biotechnology information (NCBI), and the amino acid sequence was analyzed using Clustal W2 program. Restriction enzymes of the DNA fragments in the analyzed DNA sequence were analyzed using Vector NTI advance 11 software (Invitrogen, California, USA).

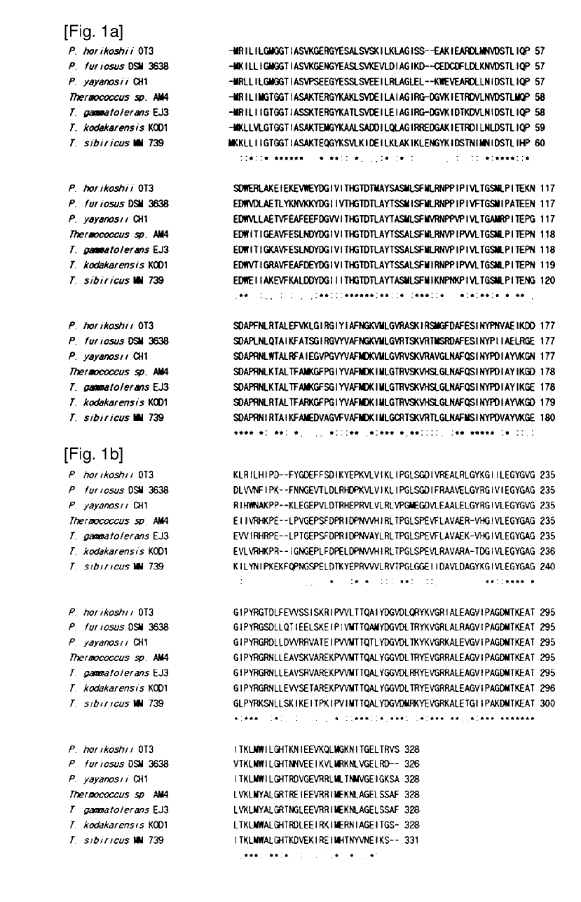

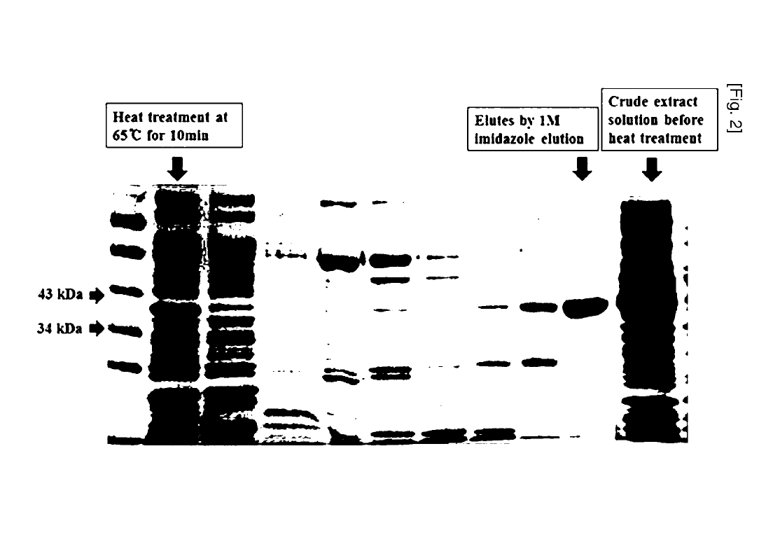

The results of analyzing the homology of L-asparaginase obtained from each strain with L-asparaginase of the present invention are shown in Table 1, FIGS. 1A and 1B.

TABLE 1

| | Size | | | Homol- |
|---|---|---|---|---|
| Accession no. | (a.a) | Predicted protein | Organism | ogy (%) |
| YP_184069.1 | 328 | L-asparaginase | T. kodakarensis | 100 |
| | | | KOD1 | |
| ZP_04879025.1 | 328 | L-asparaginase | Thermococcus | 82 |
| | | | sp. AM4 | |
| YP_002959808.1 | 328 | Glutamyl-tRNA | T. gammatolerans | 79 |
| | | (Gln) | EJ3 | |
| | | amidotransferase | | |
| | | subunit D | | |
| YP_004624668.1 | 329 | L-asparaginase | Pyrococcus | 66 |
| | | | yayanosii | |
| | | | CH1 | |
| YP_002995076.1 | 330 | L-asparaginase | Thermococcus | 63 |
| | | | sibiricus | |
| | | | MM 739 | |
| NP 142084.1 | 327 | L-asparaginase | P. horikoshii | 61 |
| | | | OT3 | |
| NP_579776.1 | 326 | L-asparaginase | P. furiosus | 58 |
| | | | DSM 3638 | |

As shown in Table 1, FIGS. 1A and 1B, it can be seen that that L-asparaginase derived from Thermococcus kodakarensis KOD1 of the present invention shows a homology of 58 to 82% with L-asparaginases derived from other strains.

### Example 4

### Purification and Characterization of Recombinant Proteins

4.1 Protein Electrophoresis and Quantification

Protein electrophoresis was performed using sodium dodecyl sulfate polyacrylamide gel electrophoresis (SDS-PAGE) according to the Laemmli method (1970). A 12% (v/v) resolving gel and a 5% (v/v) stacking gel were used. Tris-glycine SDS-polyacrylamide gel running buffer (0.025 M Tris-Cl, 0.192 M glycine, 0.1% SDS (w/v), pH 8.4) was used as a developing solvent, and the protein was mixed with SDS gel loading buffer (50 mM Tris-Cl, 100 mM dithiothreitol, 2% SDS (w/v), 0.1% bromophenol blue (w/v), 10% glycerol (v/v)) and the gel was loaded at 99° C. for 5 minutes. After the electrophoresis, the gel was stained with CBB R-250 staining solution (0.025% coomassie brilliant blue R-250 (w/v), 30% methanol (v/v), 15% glacial acetic acid (v/v)) for 1 hour, and destained with destaining solution (30% methanol (v/v), 10% glacial acetic acid (v/v)) three times for two hours each. Low protein markers (Pharmacia, Buckinghamshire, UK) containing phosphorylase b (97 kDa), albumin (66 kDa), ovalbumin, 45 kDa), carbonicanhydrase (30 kDa), and trypsin inhibitor (20.1 kDa) were used for estimation of molecular weight on SDS-PAGE. Bradford Assay (1976) was used for protein quantification.

4.2 Purification of Recombinant Proteins

To produce recombinant L-asparaginase, proteins were transformed into E. coli Rosetta (DE3) to cause excess production of protein. E. coli Rosetta (DE3) and pETK1656 were shaking-cultured at 37° C. overnight in 3 ml LB medium (containing 100 μg/ml ampicillin). The medium was inoculated with seed cultures containing E. coli 1% (v/v) grown in 1 L LB medium (containing 100 μg/ml ampicillin) and shaking-cultured at 37° C. for about 3 hours. 1 mM IPTG was added to the medium when the OD₆₀₀ reached 0.4 to 0.5 and the medium was shaking-cultured overnight. The culture liquid was centrifuged (5,000 g, 20 minutes, 4° C.), suspended in 20 ml buffer solution (20 mM sodium phosphate, 500 mM NaCl, pH 7.8), sonicated, and then centrifuged (12,000 g, 20 minutes, 4° C.).

The purified proteins were considered to have activity even at high temperatures, and the purified proteins were heated at 65° C. for 10 minutes and then centrifuged (12,000 g, 20 minutes, 4° C.). The obtained supernatant was used as a crude protein solution. During the Ni-NTA affinity chromatography, one column volume (CV) was 3 ml. The column was filled with 3 ml Ni-Sepharose FF resin and charged with 3 CV of 50 mM NiSO₄. Then, the crude protein solution was loaded onto the column such that the proteins were bound to Ni-Sepharose FF resin, and the column was washed with 5 CV of washing buffer (20 mM sodium phosphate, 500 mM NaCl, 25 mM imidazole, pH 6.8), removing non-targeted proteins. The column was eluted with 2 CV of elution buffer (20 mM sodium phosphate, 500 mM NaCl, 100 mM-1000 mM imidazole, pH 6.8), and the desired proteins were eluted and used.

Dialysis was performed to change the buffer solution of the purified protein solution. The buffer solution was 0.1M HEPES buffer (pH 8.0), and the dialysis was performed at 4° C. twice for 1 hour each using a dialysis bag from Sigma to change the buffer solution.

The produced pETK1656 was transformed into E. coli Rosetta (DE3) to express recombinant proteins, and the proteins were heated at 65° C. for 10 minutes to remove E. coli proteins produced during sonication. The proteins were purified by Ni-NTA affinity chromatography and analyzed by SDS-PAGE.

The results are shown in FIG. 2.

As shown in FIG. 2, it was found that as a result of heating at 65° C., a large amount of E. coli proteins was removed and recombinant L-asparaginase was highly purified at about 45 kDA, resulting in a single band. The single band after the purification indicates that recombinant L-asparaginase has been highly purified.

### Example 5

### Measurement of Recombinant L-asparaginase

### Activity

The activity of recombinant L-asparaginase was measured by applying the method of Shirfrin, S. (1974). L-asparaginase was added to 5 mL L-asparagine in 50 mM HEPES buffer (pH 8.0) and reacted at 90° C. for 10 minutes. Then, 15% trichloroacetic acid was added to the mixture to eliminate the protein activity, and the mixture was centrifuged at 12,000 g for 5 minutes. Then, the measurement of activity was performed at 436 nm after addition of Nessler's reagent.

5.1 Measurement of Optimal Temperature of L-asparaginase

The enzymatic reaction is very sensitive to temperature. Therefore, the change in activity of L-asparaginase with a change in temperature from 30° C. to −99° C. was measured to find an optimal temperature of recombinant L-asparaginase according to the present invention. Specifically, a mixed solution of 50 mM HEPES buffer (pH 8.0), 5 mM L-asparagine, and L-asparaginase was reacted at 30° C. to −99° C. for 1 hour.

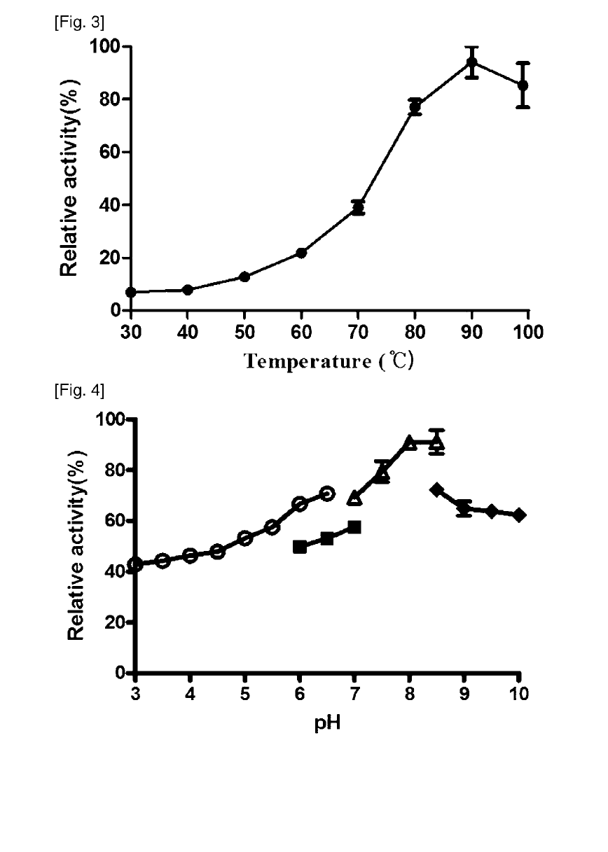

The results are shown in FIG. 3.

As shown in FIG. 3, it was found that if the activity at 90° C. exhibiting the maximum activity was taken as 100%, L-asparaginase exhibited an activity of about 80% at 80° C. and an activity of about 10% at 30° C. It was found from these results that the activity of L-asparaginase was reduced as the reaction temperature decreased.

5.2 Measurement of Optimal pH of L-asparaginase

The change in activity of L-asparaginase against pH was determined to find an optimal pH of L-asparaginase and the optimal buffer composition. Within pH range of 3 to 10, 50 mM buffer solutions such as citrate-NaOH (pH 3-6.5), sodium phosphate (pH 6-7), HEPES-NaOH (pH 7-8.5), glycine-NaOH (pH 8.5-10), etc. were added to the reaction of L-asparaginase to adjust the pH, and 5 mM L-asparagine and L-asparaginase were added and then reacted at 90° C. for 30 minutes. The pH range at which the activity was the highest was taken as 100% activity, and the relative activities were evaluated.

The results are shown in FIG. 4.

As shown in FIG. 4, L-asparaginase exhibited an activity of about 40% at a pH of 6 to 7 and exhibited the highest activity at a pH of 8. Moreover, it exhibited an activity of about 60% activity at a pH of 8.5 to 10. It can be seen from these results that the activity of recombinant L-asparaginase is maintained at a constant level even when the pH becomes either acidic or alkaline and, in particular, the activity is the highest at the neutral.

5-3 Thermal Stability of L-asparaginase

It was determined whether recombinant L-asparaginase showed stability after long-term storage at high temperatures. Recombinant L-asparaginase was placed in 50 mM HEPES buffer (pH 8.0) and heated at 60° C., 80° C. and 100° C. for 0-24 hours, and the activity was measured hourly. L-asparaginase not heated was set to 0 hour, and its activity was taken as 100% to compare the activities.

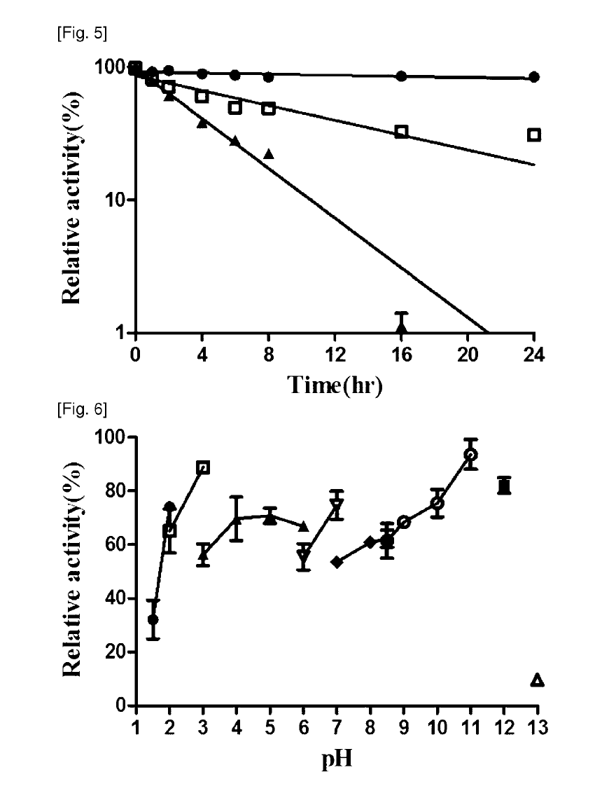

The results are shown in FIG. 5.

As shown in FIG. 5, an activity of about 90% remained even after being heated at 60° C. for 24 hours, an activity of about 50% remained even after being heated at 80° C. for 24 hours. However, it was found that the activity of L-asparaginase all but disappeared after being heated at 100° C. for 16 hours. It can be seen from these results that the Recombinant L-asparaginase of the present invention is an enzyme that has improved stability, whose activity is maintained at a high level even after long-term exposure to stress conditions such as heat.

5.4. Measurement of pH Stability of L-asparaginase

The evaluation of pH stability was performed to find the pH range where the synthesized L-asparaginase exhibits an optimal activity. The pH stability of recombinant L-asparaginase was measured according to the above activity measurement method after placing the synthesized L-asparaginase in a 50 mM solution of KCl—HCl (pH 1.5-2), glycine-HCl (pH 2-3), citrate-phosphate (pH 3-6), sodium phosphate (pH 6-7), HEPES-NaOH (pH 7-8.5), glycine-NaOH (pH 8.5-11), sodium phosphate-NaOH (pH 12), KCl—NaOH (pH 13) at 4° C. for 24 hours.

The results are shown in FIG. 6.

As shown in FIG. 6, L-asparaginase exhibited an activity of about 30% at a pH of 1.5 and exhibited an activity of about 10% at a pH of 13. At other pH levels, it exhibited relatively stable activity. It can be seen from these results that the synthesized L-asparaginase of the present invention can maintain the activity at different pH ranges and thus is highly resistant to external stress.

5.5 Effect of metal ions on L-asparaginase

In general, it can be seen that the enzymatic activity tends to increase by the effect of divalent metal ions. Accordingly, the effect of metal ions on L-asparaginase was measured by adding various metal ions to L-asparaginase. Metal ions used were CuSO₄, NiSO₄, MgSO₄, CoCl₂, ZnCl₂, CaCl₂, and EDTA (Non-addition: 1 mM, 10 mM).

The results are shown in the following Table 2.

TABLE 2

Relativity activity (%)

| Addition | 1 mM | 10 mM |
|---|---|---|
| None | 100 | 100 |
| CuSO4 | 79.38 | 0 |
| NiSO4 | 125.84 | 0 |
| CaCl2 | 89.74 | 80.16 |
| ZnCl2 | 43.78 | 28.68 |
| MgSO4 | 138.90 | 127.26 |
| CoCl2 | 69.42 | 24.57 |
| EDTA | 90.70 | 80.43 |

As shown in Table 2, it was found that while some ions decreased the activity, the activity of L-asparaginase increased about 30% when NiSO₄, MgSO₄ were added.

5.6 Measurement of Km and Vmax of L-asparaginase

The experiment was performed for each substrate concentration and for each hour to measure the substrate affinity and enzyme kinetics of L-asparaginase. Km and Vmax values were measured based on Michaelis-Menten and Lineweaver-Burk kinetics. The experiment was performed at a substrate concentration of 0 mM to 10 mM, and the activity was measured after the reaction for 0 to 20 minutes.

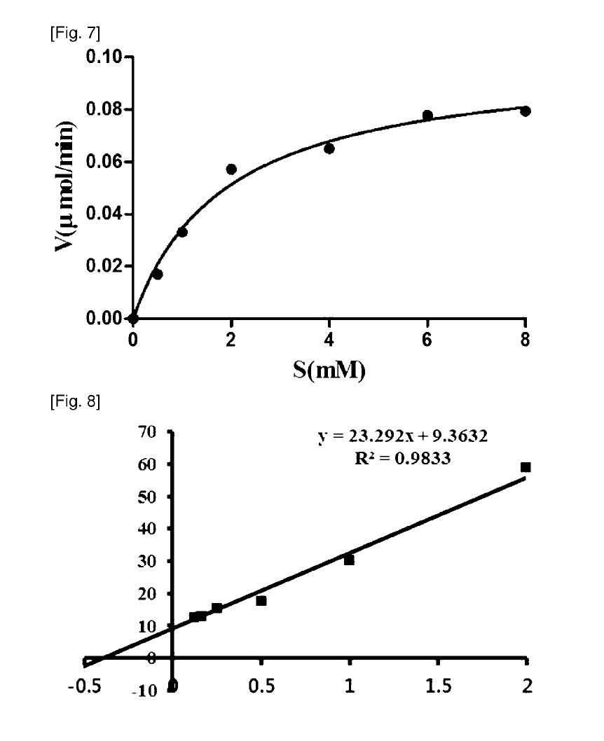

The results are shown in FIGS. 7 and 8.

As shown in FIGS. 7 and 8, the Km and Vmax values were 1.889 mM and 0.100 μmol/min, respectively.

1. A composition for breaking down L-asparagine, comprising L-asparaginase represented by an amino acid sequence of SEQ ID NO: 1.

2. The composition for breaking down L-asparagine of claim 1, wherein the L-asparaginase is derived from Thermococcus kodakarensis KOD1.

3. The composition for breaking down L-asparagine of claim 1, wherein the L-asparaginase exhibits an optimal activity at a temperature of 80° C. to 100° C.

4. The composition for breaking down L-asparagine of claim 1, wherein the L-asparaginase exhibits an optimal activity at a pH of 7 to 9.

5. A composition for breaking down L-asparagine, comprising a gene encoding an amino acid sequence of SEQ ID NO: 1.

6. The composition for breaking down L-asparagine of claim 5, wherein the gene has a nucleotide sequence represented by SEQ ID NO: 2.

7. A recombinant plasmid containing a gene having a nucleotide sequence of SEQ ID NO: 2.

8. A transformant for producing L-asparaginase, transformed with the plasmid of claim 7.

9. The transformant for producing L-asparaginase of claim 8, wherein the transformant is E. coli.

10. A method for producing L-asparaginase, comprising the steps of:
(a) culturing a transformant for producing L-asparaginase, transformed with a recombinant plasmid containing a gene having a nucleotide sequence of SEQ ID NO: 2; and
(b) isolating and purifying L-asparaginase from the transformant in step (a).

11. A method for breaking down L-asparagine, comprising the step of treating L-asparagine with L-asparaginase represented by an amino acid sequence of SEQ ID NO: 1.

12. The method for breaking down L-asparagine of claim 11, wherein the breakdown occurs at a temperature of 80° C. to 100° C.

13. The method for breaking down L-asparagine of claim 11, wherein the breakdown occurs at a pH of 7 to 9.
